(19) United States
(12) Patent Application Publication
(10) Pub. No.: US 2015/0285794 A1
(43) Pub. Date: Oct. 8, 2015

(54) HIGH-THROUGHPUT NANOIMMUNOASSAY CHIP

(71) Applicant: ECOLE POLYTECHNIQUE FEDERALE DE LAUSANNE (EPFL), Lausanne (CH)

(72) Inventors: Sebastian Maerkl, Basel (CH); Jose Luis Garcia-Cordero, San Pedro Garcia Garcia (MX)

(21) Appl. No.: 14/436,168

(22) PCT Filed: Sep. 5, 2013
(86) PCT No.: PCT/IB2013/058312

§ 371 Date: Apr. 16, 2015

Related U.S. Application Data
(60) Provisional application No. 61/715,961, filed on Oct. 19, 2012.

(51) Int. Cl. G01N 33/543

(52) U.S. Cl. CPC G01N 33/54386

(57) ABSTRACT

The nanoimmunoassay chip comprises at least flow and control layers, divided into several rows, each row containing a plurality of single assay units, each assay unit contains two spotting chambers (1) and an assay chamber in the middle, wherein neck valves (2) separate the spotting chambers from the assay chamber during surface derivatization, said assay units being isolated from one another during incubation by isolation valves (3), wherein relief valves (4) help release built-up pressure into a microfluidic channel (5) after incubation and wherein round valves in the assay chamber define and protect the circular immunoassay regions (6).

### FIELD OF THE INVENTION

The present invention concerns a multiplexed high-throughput nanoimmunoassay microfluidic device capable to quantify four biomarkers in 384 5-nL biological samples for a total of 1,536 assays.

The sample throughput of the chip according to the invention is 30 times higher than recent integrated microfluidic systems (Heath et al, Nat Biotech, 2008, and Huang et al, Lab Chip, 2012), with an order of magnitude higher assay throughput. This ultra high-throughput translates into a 1,000 fold reduction in reagent costs and a significant reduction in personnel cost per sample leading to a highly competitive diagnostic tool as compared to standard ELISA and/or multiplexed ELISA. The limit of detection is 100 fM, a similar performance as ELISA, but does so by detecting as little as 600 antigen molecules in 5-nL volume samples (˜1 zeptomole), 20-fold lower than current state-of-the-art techniques (Duffy et al, Nat Biotech, 2010).

The chip according to the invention is compatible with a number of complex biological matrices/samples including, but not limited to, blood serum, cell culture medium, and bronchoalveolar lavage (BAL). In one application, our nanoimmunoassay chip enabled a large-scale screening study by reducing the cost of reagents for the experiment from 20,000 Euros down to 15 Euros, and by automating and streamlining the entire process.

More generally, the nanoimmunoassay chip according to the invention will have a significant impact on the healthcare sector by drastically reducing the cost of diagnostic assays. In fact, in the near future it will be possible to routinely and periodically screen small blood samples from healthy individuals for large panels of disease indicators. With technologies such as the nanoimmunoassay chip described here, the cost of such preventative screens will be minimal, and be far outweighed by the benefits and cost reductions associated with early diagnosis of disease. Additionally, low-cost diagnostics will give rise to personalized diagnostics. In personalized diagnostics many hundreds of biomarkers are expected to be measured in short intervals (a few times a year) per individual. This wealth of data will generate a personalized base-line indicative of health, and allow the identification of departures from normalcy.

### BACKGROUND OF THE INVENTION AND PRIOR ART

While the most recent commercial technologies have drastically increased biomarker throughput, these novel immunoassays bear similar drawbacks as their classic counterpart, the enzyme-linked immunosorbent assay (ELISA), such as requirement of large sample volumes, long process and hands-on times, poor automation, and high costs. The integration of microfluidics with micro/nano-scale biosensors has been touted for over 15 years as a solution to these technical challenges, not only by reducing sample volume and reagents consumption, but also by decreasing limits of detection (LOD), offering multiplexing, automation, and systems integration, while keeping the overall system simple in design and low in cost^{1, 2}. However, the most sensitive biosensors, based on beads, require large sample volumes not suitable for miniaturization; moreover, complex fabrication, surface bio-functionalization issues, and desalting steps in nanowire and MEMS biosensors render the integration of microfluidic devices with the above mentioned characteristics challenging and costly. Here, we describe an integrated and automated multiplexed nanoimmunoassay chip, which combines advantages of microfluidics and microarray technologies, delivers performances similar to ELISA, and at the same time drastically reduces sample and reagent volume consumption and cost 1000-fold, while drastically increasing sample throughput.

### DETAILED DESCRIPTION OF THE INVENTION

The present invention will be better understood from the following detailed description and from appended drawings which show:

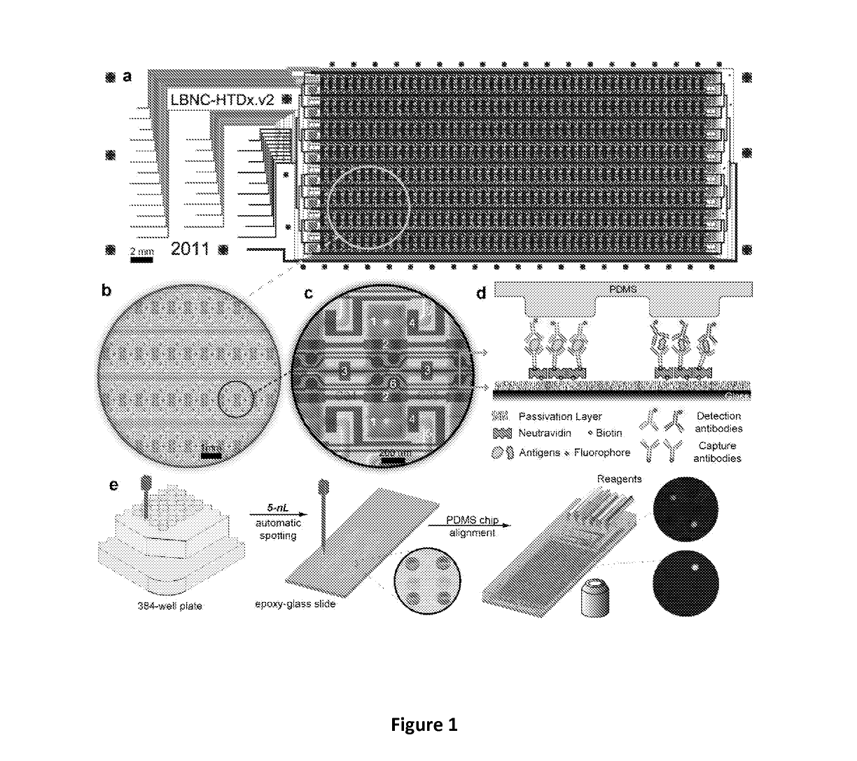

FIGS. 1a to 1c illustrate the principle of the chip of the invention;

FIG. 1d illustrates an embodiment of an immunoassay;

FIG. 1e illustrates the principle of a method according to the present invention;

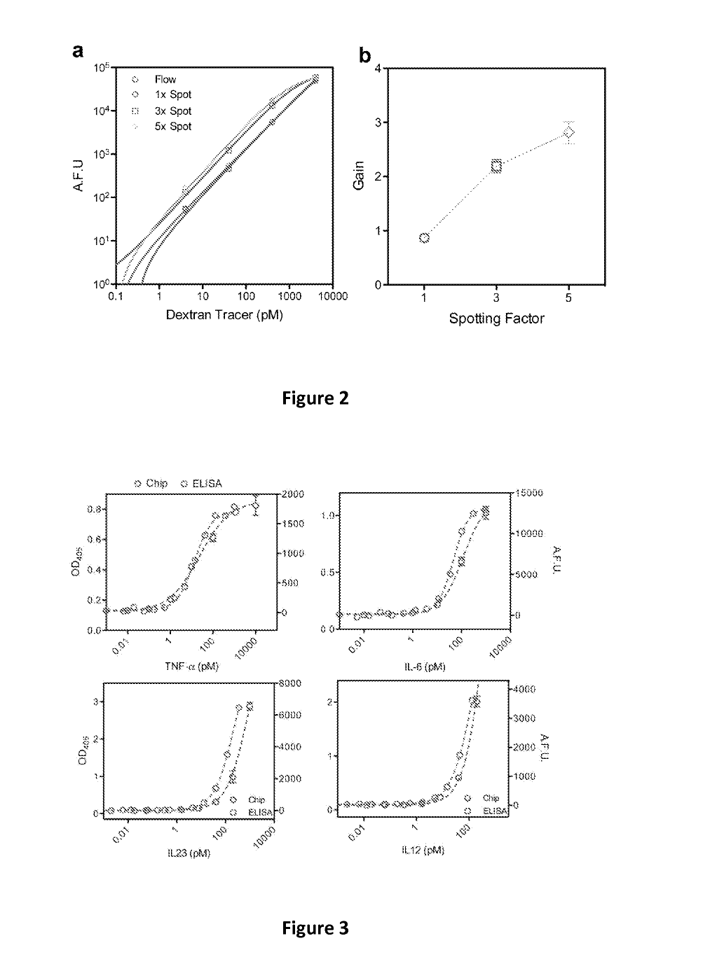

FIG. 2 illustrates immunoassay performance characterization using a fluorescent tracer;

FIG. 3 illustrates a comparison of calibration curves for TNFα, IL-6, IL-12, and IL-23 obtained with the nanoimmunoassay chip of the invention and a typical ELISA;

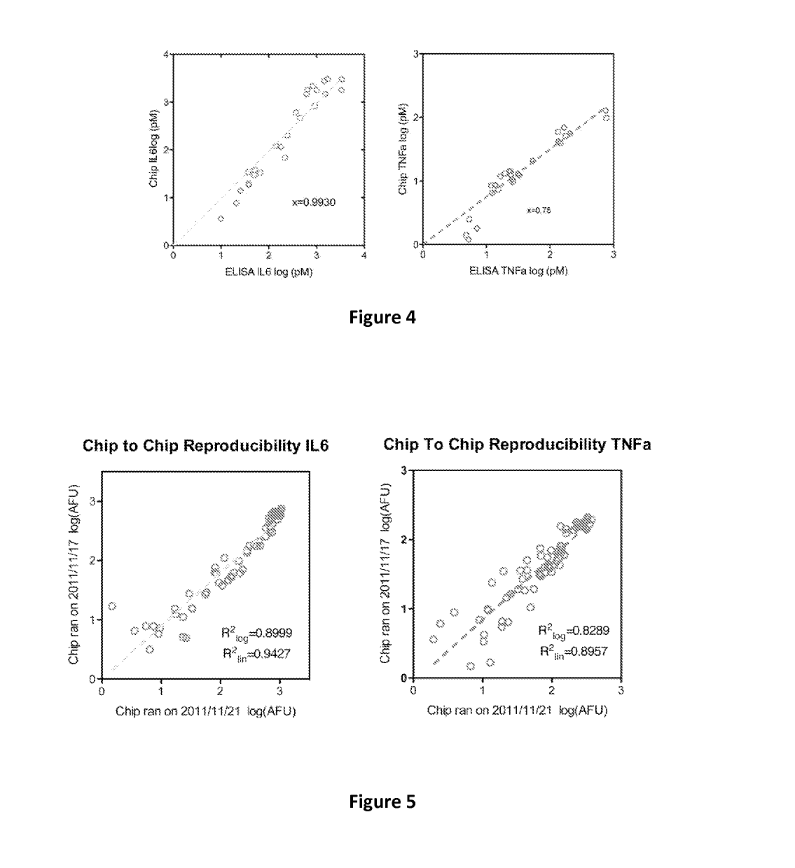

FIG. 4 illustrates a comparison of blind tests run with the nanoimmunoassay chip and an ELISA;

FIG. 5 illustrates chip to chip reproducibility over time.

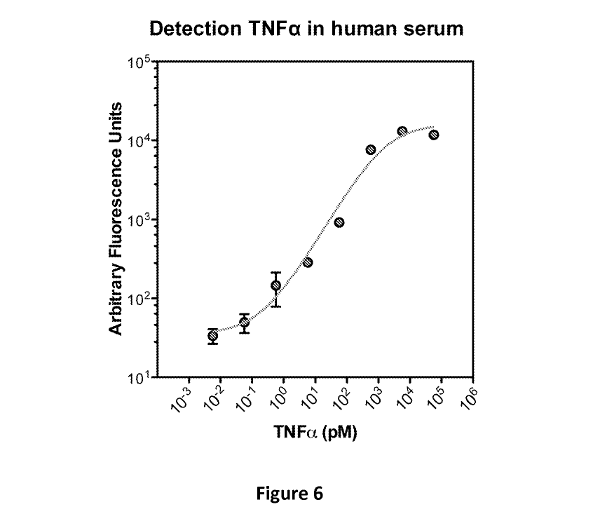

FIG. 6 illustrates detection of biomarker TNFα in human serum.

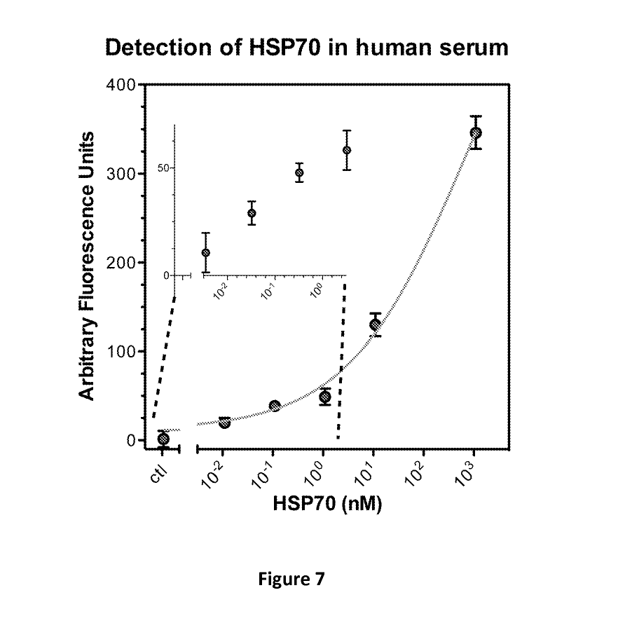

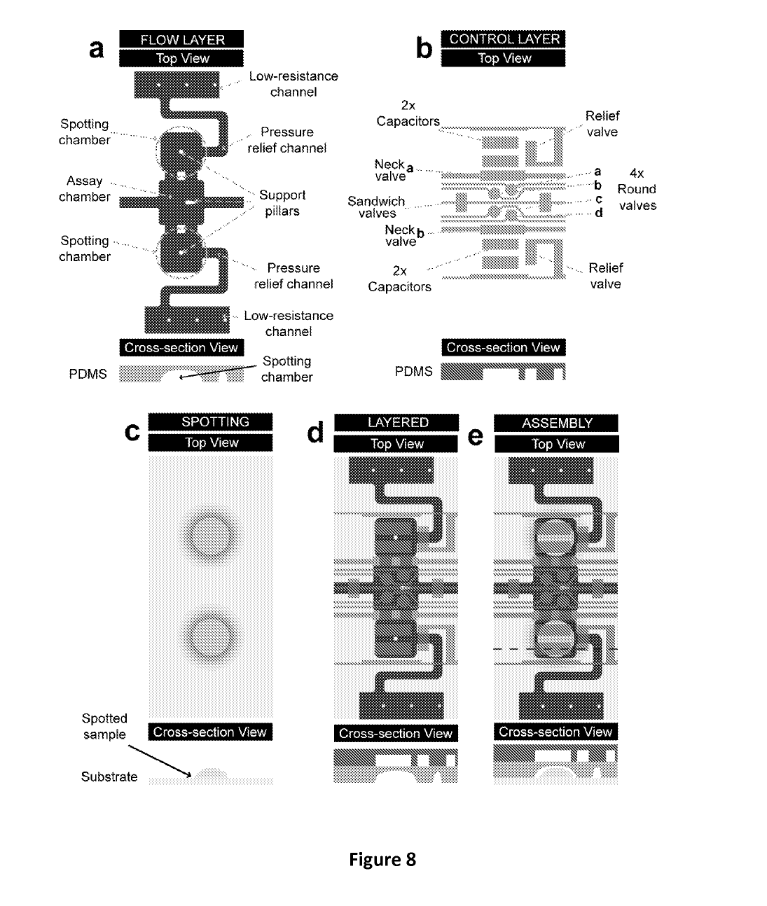

FIG. 7 illustrates detection of biomarker HSP70 in human serum.

FIG. 8 illustrates a single assay unit;

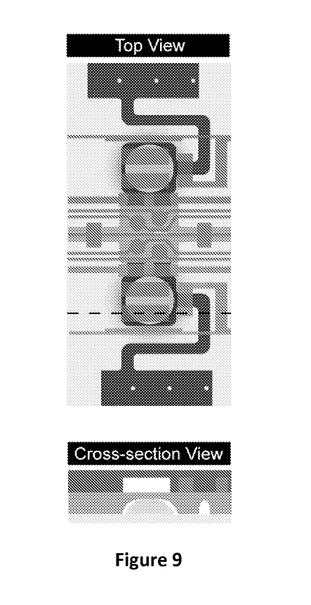

FIG. 9 illustrates a passivation step;

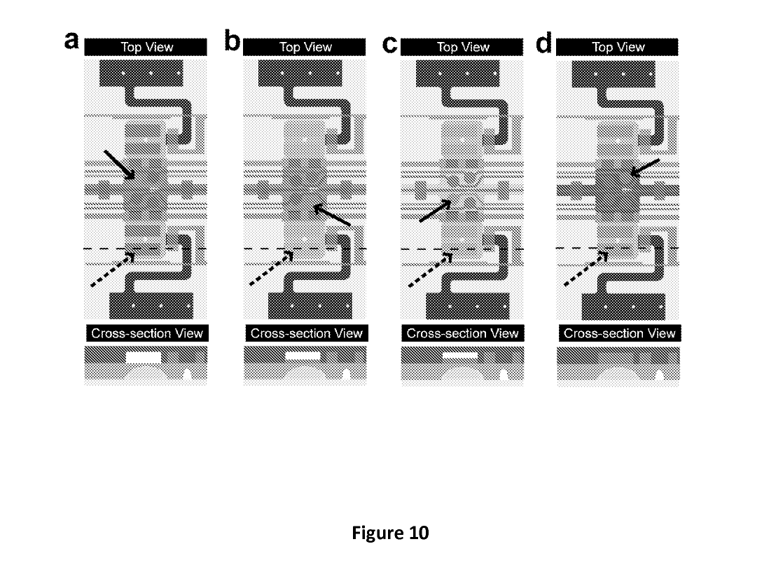

FIG. 10 illustrate an antibody immobilization step;

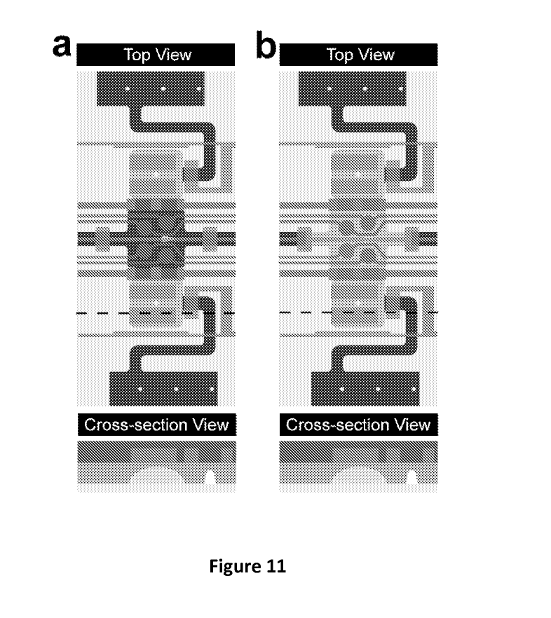

FIG. 11 illustrates an Incubation step;

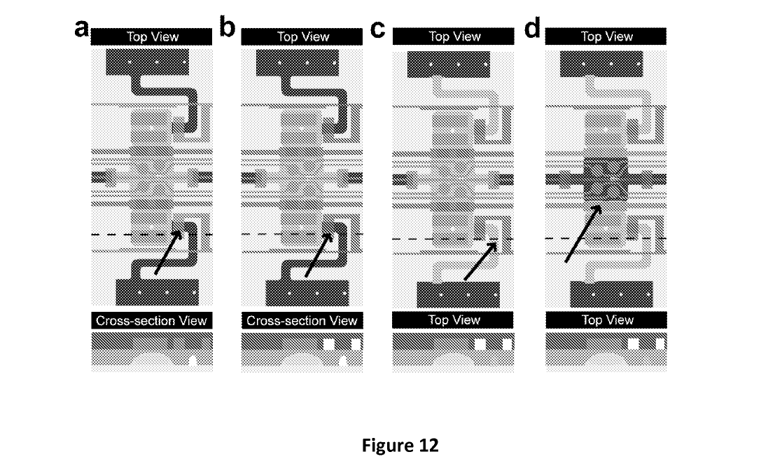

FIG. 12 illustrates a washing step;

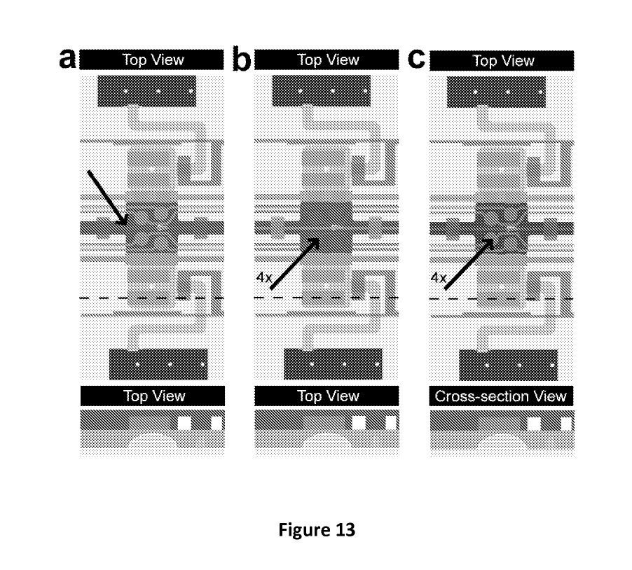

FIG. 13 illustrates a washing step.

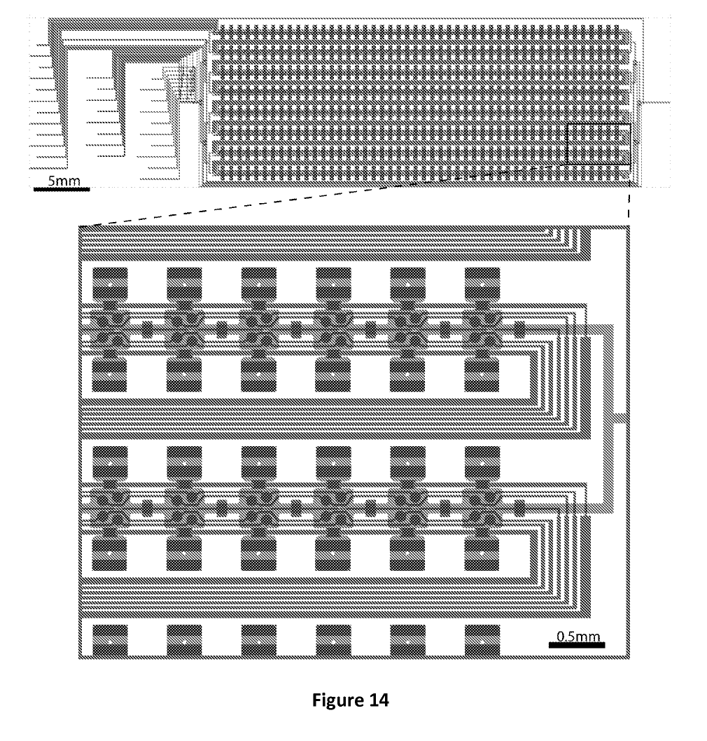

FIG. 14 illustrates an alternative microfluidic device design.

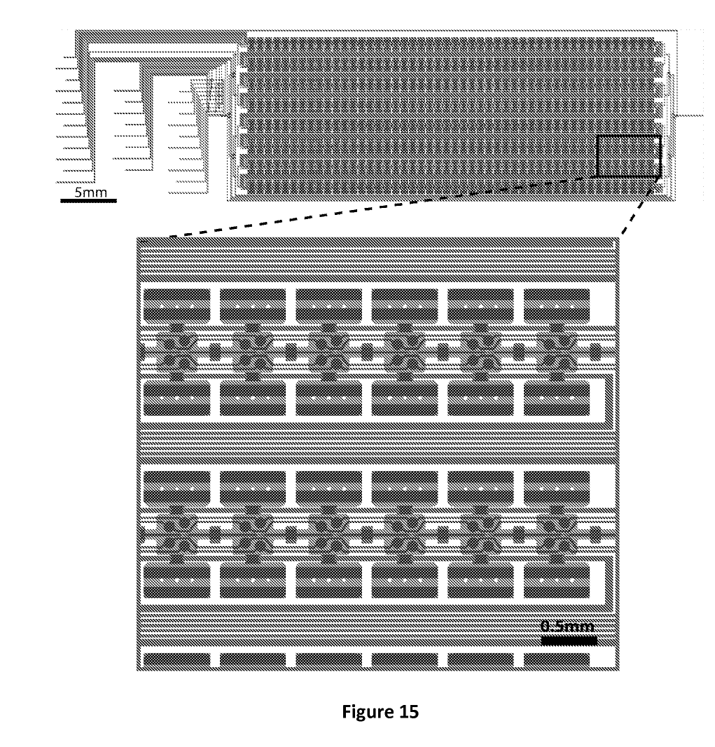

FIG. 15 illustrates an alternative microfluidic device design.

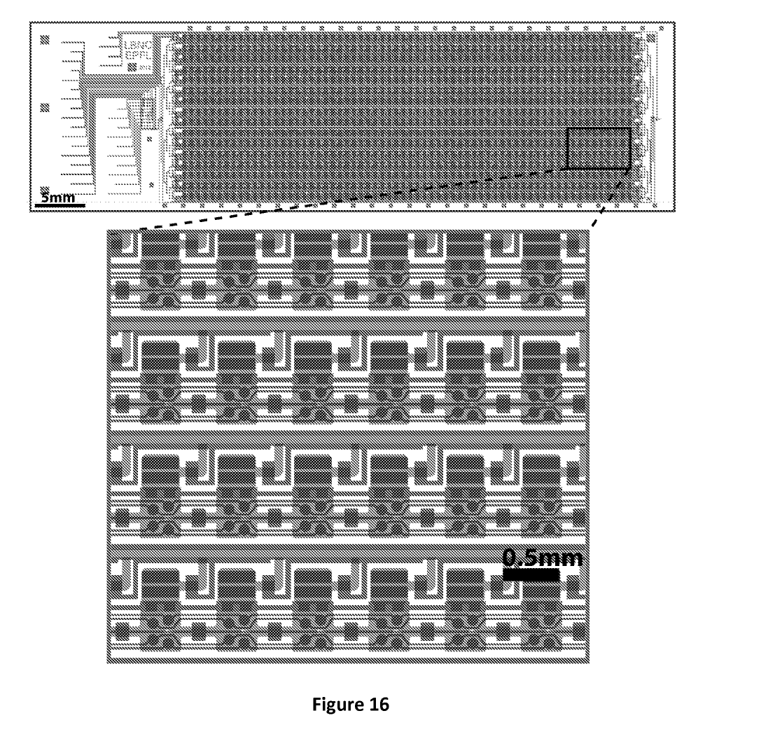

FIG. 16 illustrates a microfluidic device design with 1024 chambers.

The nanoimmunoassay chip according to the invention is capable of analyzing 4 biomarkers in parallel from 384 biological samples using nanoliter volume samples, for a total of 1,536 measurements per chip.

The platform is based on a polydimethylsiloxane (PDMS) microfluidic chip of 384 assay units (FIGS. 1a, b). Each assay unit contains two 1.7-nL spotting chambers that encapsulate the same sample (FIG. 1c). Assay units are isolated from one another during incubation steps with isolation valves to eliminate cross-contaminations. A 1-nL reaction chamber, which lies between the spotting chambers, contains four circular immunoassay regions of 60-μm diameter created in situ by rounded valves using a mechanism developed by Maerkl et al. Any biotinylated capture antibody can be immobilized in these regions, allowing for the parallel detection of four biomarkers of choice (FIG. 1d). Samples to analyze are automatically picked from a 384-microtiter plate with a microarray robot, and precisely spotted on an epoxy-functionalized microscope glass slide using a 5-nL delivery-volume spot pin (FIG. 1e). The PDMS chip is then directly aligned on top of the spotted slide and bonded. After derivatization of the chip surface, and immobilization of the biotinylated capture antibodies in the reaction chambers, the rehydrated spotted sample is allowed to diffuse and react with the capture antibodies. Detection occurs after flowing a fluorescently-labeled secondary antibody.

For a more detailed description of the fabrication and operation of the device see Device Fabrication and Operation sections below.

Specifically, FIG. 1 illustrates a nanoimmunoassay chip workflow.

FIG. 1(a) The microfluidic device comprises flow (blue) and control (red) layers, divided into eight rows

FIG. 1(b) each row containing 48 single assay units for a total of 384 units.

FIG. 1(c) Each assay unit contains two spotting chambers (1) and an assay chamber in the middle. Neck valves (2) separate the spotting chambers from the assay chamber during surface derivatization. Assay units are isolated from one another during incubation by isolation valves (3). Relief valves (4) help release built-up pressure into a microfluidic channel (5) after incubation. Four round valves in the assay chamber define and protect the circular immunoassay regions (6).

FIG. 1(d) A sandwich immunoassay is performed under each round valve with a combination of biotinylated and fluorophore-labelled antibodies.

FIG. 1(e) Biological solutions kept in a microtiter well-plate are automatically spotted onto an epoxy-coated glass slide using a microarray robot. Dried spots have a diameter of ˜350 μm. A microfluidic chip made by multilayer soft-lithography is aligned on top of the spotted slide. Different reagents are loaded into plastic tubing and connected to the chip. A fluorescent scanner reads the fluorescent intensity of the immunoassay regions.

In contrast to a 96-well plate ELISA that requires coating of each well with 50-400 ng of antibody, each assay unit on-chip according to the present invention requires 20-160 pg. This corresponds to a decrease in the amount of antibody needed of more than three orders of magnitude for similar number of assays (see Table 1). Overall, the nanoimmunoassay chip according to the invention reduces cost and sample volumes by at least a factor of 1,000 while offering complete integration and automation with minimum user intervention (see Table 1).

TABLE 1
Comparison of nanoimmunoassay chip and a 96-well plate
ELISA per single assay unit

| | Nanoimmunoassay | | | |
|---|---|---|---|---|
| | chip | | ELISA | |
| Effective assay volume | 5 | nL | 100 | uL |
| Sample volume | 10 | nL | 100 | μL |
| Capture antibody amount | 20-160 | pg | 50-400 | ng |
| Detection antibody amount | 20-160 | pg | 50-400 | ng |
| Standard protein volume | 10 | nL | 100 | uL |
| Enzymatic amplification step | No | | Yes | |
| Multiplexing | 4 | | 1 | |
| LOD (TNFα, IL6) | 100 | fM | 100 | fM |
| Hands-on time | 10 | min | 100 | min |
| Automation | Microfluidics | | Robot | |
| Pipetting steps | 1 | | 30 | |
| Type of samples | Various (culture media, serum, BAL) | | | |
| Total reagent consumption volume | 0.5 | μL | 7700 | uL |

| | | |
|---|---|---|
| Total cost of reagents | ~$0.005-0.020 | ~$5-20 |

The performance of the chip is assessed by quantifying the amount of protein effectively diffusing from the spotting chambers into the reaction chambers. We spiked different concentrations of a fluorescent tracer (Alexa647-labeled Dextran, 10 KDa) in undiluted serum and spotted the solutions onto the chip. The same solutions were flowed into the chip and fluorescent intensity values were compared to the spotted values. We observed a 100% recovery of the tracer into the reaction chambers (FIG. 2a); moreover we found that multi-spotting allows for up to three-fold higher sample concentration (FIG. 2b).

More specifically, FIG. 2 illustrates Immunoassay performance characterization using a fluorescent tracer. Different concentrations of a fluorescent tracer (Alexa647-labeled Dextran 10 KDa) were diluted in serum and spotted onto the chip. The same solutions were flowed onto the chip and fluorescent intensity values were compared to the spotted values. A 100% reconstitution of the tracer into the reaction chambers was observed (FIG. 2a). As up to 93% of blood serum consist of water, we reasoned that spotting multiples times with intermittent pauses to allow evaporation, could lead to sample concentration and thus increase the amount of protein spotted. Multi-spotting allows for up to three fold sample concentration and thus three times higher protein concentrations can be gained by multi-spotting five times onto the same position (FIG. 2b). Higher multi-spotting numbers are limited by the size of the microfluidic assay units, nevertheless this technique demonstrates to be a simple alternative to other microfluidic pre-concentration methods.

As a second step, the sensitivity of our platform was determined by running calibration curves for the cytokines IL-6, TNFα, IL-12p70, IL-23 in cell culture medium; LOD and dynamic range were comparable to ones obtained with ELISA (FIG. 3). Notably, for the lowest concentration detected, 100 fM, the platform of the invention is able to readily detect 830 molecules (˜50 zeptomoles) using the same antibody combinations used in commercially available kits. Thus, the present microfluidic approach for biomarker detection compares favorably with other biosensors in terms of sensitivity but surpasses them in terms of simplicity and throughput.

FIG. 3 illustrates a comparison of calibration curves for TNFα, IL-6, IL-12, and IL-23 obtained with the nanoimmunoassay chip of the invention and a typical ELISA. Calibration curves for cytokines IL-6, TNFα, IL-12p70, IL-23 in cell culture media were spotted and found to be similar to ones obtained with a standard ELISA.

To further compare the chip to standard ELISA methods, we determined the concentration of IL-6 and TNFα in stimulated cell culture samples known to express both cytokines at a wide concentration range. Samples were analyzed on-chip in triplicates, including 8 known protein dilutions for the calibration curves of each cytokine, as well as 10 blank controls, for a total of 234 spotted and 428 data points. A log correlation of 0.96 and 0.75 was found for IL-6 and TNFα, respectively, (FIG. 4), demonstrating that the nanoimmunoassay chip according to the invention is as accurate as an ELISA. To evaluate the stability, reproducibility, and robustness of our platform over time, a chip spotted on the same day was run five days later and similar correlations were found (FIG. 5).

FIG. 5 illustrates Chip to chip reproducibility over time. The chip used to determined the concentration of unknown cell culture samples was run five days later and log correlations of 0.89 and 0.82 for IL-6 and TNFα, respectively, were observed.

FIG. 6 illustrates detection of biomarker TNFα spiked in human serum at different concentrations. The limit of detection is 570 fM (defined as 3 times the standard deviation of the control signal).

FIG. 7 illustrates detection of biomarker HSP70 spiked in human serum at different concentrations. The limit of detection is 10 pM (or 800 pg/mL), which compares favorably with commercial ELISA kits that have a limit of detection of 780 pg/mL (HSP70 ELISA Kit, ADI-EKS-700B, Enzo Life Sciences).

### Device Fabrication

### 2.a. Chip Fabrication

The microfluidic device comprises two layers. Molds for each layer were fabricated using standard lithography techniques on 4″ silicon wafers. Briefly, photolithography masks were laid out in Clewin (WieWeb, Netherlands) and photo-plotted on a chromium substrate pre-coated with AZ1518 (Nanofilm, CA) using a laser pattern generator (DWL2000, Heidelberg Instruments, Germany). The control and flow layer molds were patterned with SU8 phothoresist (GM1060, Gersteltec, Switzerland) to a height of ˜30 μm, and with AZ9260 photoresist (Microchemicals, Germany) to a height of ˜10 μm, respectively, according to manufacturer instructions. The flow layer mold was baked for 2 hours at 180° C. to reflow the photoresist and obtain rounded structures. Molds were treated in a vapor bath of trymethylchlorosilane (TMCS, Sigma-Aldrich, USA) for 30 min before using them.

Devices were cast in polydimethylsiloxane (Sylgard 184, Dow Corning, USA) employing different ratios of curing agent. The control layer was cast thick (˜5 mm) using a ratio of 1:5 and degassed for 10 min in a vacuum desiccator. PDMS, at a ratio of 1:20, was spun on the flow layer mold at 2100 rpm in a spin coater (P6700, Specialty Coating Systems, USA) to obtain a thin layer (˜30 μm). Both molds were baked at 80° C. for 30 min in a convection oven. Next, the PDMS control layer replicas were peeled-off from the mold and holes for control ports punched using a manual hole-puncher. The control replicas were manually aligned on top of the flow layer and baked at 80° C. for 1.5 hours. Aligned replicas were cut and peeled-off from the mold. Finally, holes for flow inlet ports were manually punched.

Microfluidic flow and control pressure regulation was achieved using a custom built pneumatic setup. Pressure for flow lines was set to 3 psi using an analog pressure gauge. Microfluidic control lines were grouped in two sets, one set for the microfluidic rounded valves and the other set for the rest of the control lines. Each set was connected to two different pressure gauges through a 3-way solenoid valves (Pneumadyne Inc). Solenoid valves were controlled from a PC by means of a graphical using interface programed in LabView.

### 2.b Preparation of Epoxy-Silane Glass Slides

This protocol for coating glass slides, adapted from Nam et al¹, produces a homogenous, dense monolayer of epoxy-silane groups on the surface of the glass, where epoxy groups are preferentially exposed on the surface of the monolayer. Glass slides were functionalized as follows. A solution of 720 mL of milli-Q water and ammonia solution (NH₄OH 25%, 1133.2500, VWR) in a 5:1 ratio, respectively, was heated to 80° C. Next, 150 mL of hydrogen peroxide (H₂O₂ 30%, 99265, ReactoLab, Switzerland) were added to the mix and cut-edge glass microscope slides (631-1550, VWR) bathed in the solution for 30 min. Glass slides were then rinsed with milli-Q water and blow-dried.

A solution of 1% 3-Glycidoxypropyl-trimethoxymethylsilane (97% pure, 216545000, Acros Organics) in toluene was prepared and the glass slides incubated in it for 20 min. Glass slides were then rinsed with toluene and blow-dried, followed by a baking step for 30 min at 120° C. The glass slides were sonicated in toluene for 20 min, rinsed with isopropanol, and N₂ blow-dried. Finally, glass slides were vacuum-stored at room temperature.

### 2.c Automatic Sample Microarraying

Biological samples were pipetted into a 384-well microtiter plate (No. 264573, Thermo Fisher Scientific, USA). Samples were spotted in triplicate onto epoxy-silane coated glass slides using a microarray robot (QArray2, Genetix, UK) with a 4.9 nL delivery-volume spot pin (946MP8XB, Arrayit, USA). It is possible to spot up to 48 samples in parallel with a similar number of pins. A glass slide can contain a maximum of 768 spots (2 spots per assay). Samples were randomly spotted on glass slides; up to three slides were spotted on one round.

The humidity of the microarray robot chamber was set to 60%. We found that 60% humidity gave us the most consistent features in terms of spot diameter (˜300 μm). This humidity percentage also prevented the sample channel of the spotting pin to dry and therefore get clogged. For viscous samples, such as serum, we found that using the Touch Off feature on the robot reduced blotting—remove excess sample from the pin tip. A 2-step Touch Off with a 500 msec pause after dipping was found sufficient. A stringent wash between spotting different samples was necessary to prevent any carry-over from sample to sample. The table 2 below shows the sequence of washing steps we found were adequate to avoid cross-contamination.

TABLE 2

| Liquid | Wash time (sec) | Dry time (sec) |
|---|---|---|
| De-ionized water | 5 | 5 |
| De-ionized water | 5 | 5 |
| PBS/0.05% Tween 20 | 3 | 3 |
| De-ionized water | 5 | 5 |
| PBS/0.05% Tween 20 | 3 | 3 |
| De-ionized water | 5 | 5 |

Spotted slides were stored in the dark for at least two hours in an incubator at 40° C. before manual alignment of the PDMS device. For high-humidity environments, this step allowed for most of the water to evaporate from the sample and thus facilitate device alignment. The assembled device was incubated overnight in the dark at 40° C.

### 2.d Antibodies and Recombinant Cytokines

Mouse antibodies and standard proteins used, all purchased from eBioscience (San Diego, USA), are summarized in the table below. Purified primary antibodies for IL-23p19 and IL-12p35 were purchased, and subsequently biotinylated using a biotinylation kit (EZ-Link Micro Sulfo-NHS-Biotinylation Kit, Thermo Fisher Scientific, Rockford, USA) according to the manufacturer instructions. All mouse secondary antibodies were conjugated with phycoerythrin (PE). We used a common secondary antibody for the detection of IL-12 and IL-23 that reacts with the p40 subunit of both antibodies.

TABLE 3

| | Mouse | Capture antibody | Detection antibody |
|---|---|---|---|
| | recombinant protein | (Biotin) | (PE) |
| IL-6 | 39-8061-60 | 36-7062-85 | 12-7061-41 |
| TNF-alpha | 39-8321-60 | 13-7341-81 | 12-7423-41 |
| IL-23 | 39-8231-60 | 16-7232-85 | 12-7123-41 |
| IL-12 p70 | 39-8121-60 | 14-7122-85 | 12-7123-41 |

### 3. Device Operation

The platform is based on a polydimethylsiloxane (PDMS) microfluidic chip of 384 assay units fabricated by multilayer soft-lithography as described in the previous section. A single assay unit consists of flow and control layers (FIGS. 8.a, b). The flow layer consists of an assay chamber and of two spotting chambers that encapsulate the dry spotted biological solutions (FIG. 8.a). The spotting chambers contain a pressure relief channel that terminates in a low resistance fluidic channel. Support pillars in the different chambers prevent the PDMS roof from collapsing into the substrate. The control layer (FIG. 8.b) includes 4 round valves that overlap with the assay chamber. Two neck valves isolate the spotting chambers from the assay chamber. Two sandwich valves isolate single assay units from one another during incubation steps. To help release some of the build-up pressure that occurs during rehydration of the spotted chambers, relief valves are open for the pressurized fluid to flow through the pressure relief channel into the low-resistance channels. Biological solutions are spotted on a planar substrate (FIG. 8.c) and align with the assembled chip (FIGS. 8.d, e).

FIG. 8 illustrates a single assay unit schematic.

FIGS. 8(a, b) Each assay unit comprises two layers fabricated by multilayer soft-lithography.

FIG. 8(c) The biological solution is spotted twice on a planar substrate

FIGS. 8(d, e) aligned with the assembled assay unit and

Execution of nanoimmunoassay chip protocol

a. Reagent loading. All reagents were aspirated into Tygon tubing (0.020″ ID, AAQ02103, Coler-Parmer). 80 μL of PBS buffer with 0.05% Tween-20 was connected to the first inlet of the device. PBS/Tween was used as a washing buffer throughout the experiments. 30 μL of biotinylated BSA (29130, Thermo Fisher Scientific) at a concentration of 2 mg/mL and 15 μL of neutravidin (31000, Thermo Fisher Scientific) at 0.5 mg/mL were connected to the second and third inlet, respectively. 10 μL of 5% milk powder resuspended in PBS was connected to the fourth inlet.

b. Control line priming. Microfluidic control channels were primed with dH₂0 at 6 psi. Once the channels were filled the pressure was increased to 20 psi to close all the valves except for the rounded valve lines (FIG. 9).

c. Biotin-neutravidin layer deposition. Reaction chambers were passivated by flowing biotin-BSA for 20 min at 3 psi. At this step, it is possible to use blocking buffers such as BSA, milk, or casein while keeping the buttons closed but this adds another step and consequently increases the assay time. Biotin-BSA was washed by flowing PBS/Tween for 5 min. Neutravidin was then flowed for 20 min through the chambers and washed for 5 min. The pressure in the rounded valve lines was increased to 20 psi and the rounded valves closed. Closing the rounded valves mechanically shields a round area of ˜2700 μm² (60-μm diameter) at the bottom surface and delineates the space where the sandwich immunoassay takes place. Biotin-BSA was flowed again for 20 min followed by a washing step of 5 min. Next 5% of non-fat dry milk in PBS was flushed for 10 min and washed for 5 min.

FIG. 9 illustrates a. Passivation step. Relief and neck valves are closed and different reagents required for the passivation step flowed through the assay chamber.

d. Primary antibody immobilization. Each primary antibody is immobilized under its corresponding rounded valve, FIG. 10. Biotinylated antibodies were diluted in 1% blocker casein in PBS (37528, Thermo Fisher Scientific). Optimal working concentration for all primary antibodies was found to be 2 μg/mL except for anti-IL6 antibody, which was 200 ng/mL. 15 μL of each antibody dilution were loaded into different Tygon tubing pieces and connected to the device. (At this step there is a layer of biotin-BSA-neutravidin under the area protected by the rounded valves.) One of the rounded valves was opened while keeping the rest of the rounded valves closed, and the first antibody was flowed for 20 min followed by a 10 min washing step. This process was repeated for the remaining primary antibodies. A final blocking step with milk was performed by opening all the valves, flowing milk for 10 min, and finally washing for 5 min with PBS/Tween.

Water from the control lines diffuses constantly through the PDMS due to is porosity, thus all spotted samples have fully rehydrated at this step. This also increases considerably the pressure inside the spotting chambers. A couple of capacitor strategically located on top of the spotting chambers release some of this pressure by absorbing some of the water from the pressurized spotting chamber.

FIG. 10 illustrates an Antibody immobilization step. Rounded valves are open sequentially to immobilize different antibodies under each of them. Solid arrows point to the different rounded valves closed during each step. Dotted arrows point to the spotting chambers. Over time, the spotted solution rehydrates because of water permeation through the PDMS from pressurized valves and builds up pressure in the chamber. A couple of capacitors sitting on top of the chambers help release some of this pressure by water permeation through the membrane separating the control layer and the flow layer.

e. Sample incubation. To incubate the sample, the isolation valves separating each reaction chambers were closed and the chamber valves opened, FIG. 11. Rehydrated samples are incubated for at least one hour at room temperature.

FIG. 11 illustrates an Incubation step. (a) Sandwich valves are closed to isolate single assay units from each other. (b) The neck valves are opened and the rehydrated sample diffuses through the chambers and allowed to incubate.

f. Sample washing. During incubation the pressure across the three chambers equilibrate (the pressure is higher in the spotting chamber than the reaction chamber before incubation), raising the reaction chamber internal pressure to the point were the rounded valves will not close when actuated. Relief valves are opened to dissipate some of the pressure in the spotting chambers (and therefore in the reaction chamber) and to allow the rounded valves to fully deflect and protect the antibody-antigen complex (FIG. 12). After a few seconds the chamber valves are closed, isolation valves open, and unbound material washed away by flowing PBS/Tween for 20 min.

FIG. 12 illustrates a. Washing step.

FIGS. 12(a, b) After incubation, round valves are closed and relief valves are opened to release some of the pressure, arrows pointing to bottom relief valve.

FIG. 12(c) After a few seconds some of the biological solution overflows into the relief channel.

FIG. 12(d) Next, the neck valves are closed, sandwich valves opened and the assay chamber washed.

g. Detection step. A cocktail of secondary detection antibodies was diluted in casein/PBS to a concentration of 0.01 μg/mL, 0.05 μg/mL, and 1 μg/mL, for IL6, TNFα, and IL-12/IL-23 p40 antibodies, respectively. The cocktail was flowed through the chip for 10 min, isolating valves closed, and rounded valves opened. After the secondary antibodies were incubated with the bound complex for 20 min, the rounded valves were closed to protect the sandwich complex, followed by a final wash of PBS/Tween for 10 min to remove unbound antibodies.

FIG. 13 illustrates a further washing step.

FIG. 13(a) A cocktail of detection antibodies is flowed through the chip. Arrow points to the assay chamber.

FIG. 13(b) Next, sandwich valves are closed and all the rounded valves open; arrows pointing to the rounded valves. Detection antibodies are bound to their respective antigens.

FIG. 13(c) After 15 min, rounded valves are closed again and sandwich valves open. A final washing step is performed.

h. Optical Readout. The microfluidic device was scanned using a fluorescent microarray scanner (ArrayWorx e-Biochip Reader, Applied Precision, USA) equipped with a Cy3 filter (540/25 X, 595/50 M). Devices were scanned with an exposure time of 1 sec at the highest resolution of 3.25 μm. Stitched images were exported as a 16-bit TIFF file.

i. Data analysis. Image files were analyzed using a microarray image analysis software (GenePix Pro v6.0, Molecular Devices) and Matlab (Mathworks). An analysis template grid was manually created containing 1536 circular features that matched the location of the 4 rounded valves for the 386 reaction chambers on the chip. For each chip, the grid was manually aligned and the diameter of the circular features adjusted for each of the 1536 detection assays. Although this step could be automated, the manual alignment of the PDMS device to the glass slide introduced an inconsistent offset between rows and columns that varied across chips. The positions of each feature and its diameter were saved in a text file, which was fed to a Matlab script. The script automatically computes the mean fluorescent intensity inside each feature and subtracts the local background around the feature. It then generates a file reporting the relative fluorescent unit (RFU) values of the four different biomarkers for each assay. The script also arranges the RFU values of the calibration curves for each biomarker.

l. Statistics Analysis. A statistical software (Prism v5.0, GraphPad) was used to perform a non-linear regression analysis on the standard curves. Data from the calibration curves were fit using a dose-response model (variable slope, four-parameters), weighting data points by the observed standard deviation. Unknown RFU values were interpolated from the standard curves.

### 4. Alternative Microfluidic Device Designs

Other microfluidic device designs as illustrated in FIG. 14, FIG. 15, and FIG. 16, can be used to perform the same operations described here. FIG. 14 illustrates a device similar to the one described in FIG. 1 but without a relief valve. FIG. 15 illustrates a device with bigger spotting chamber to increase assay sensitivities. FIG. 16 illustrates a device capable to perform 1024 nanoimmunoassays in parallel.

### 5. Materials and Reagents Used

### Bone Marrow-Derived Dendritic Cell Culture

Bone marrow-derived dendritic cells (BM-DCs) were generated as previously described (ref Lutz). Briefly, bone marrow cells were flushed from the femur and tibiae of 7 weeks old C57BL/6 mice and cultured for 9 days in RPMI medium (Invitrogen/LuBioScience, Lucern, Switzerland) supplemented with 10% FBS, Penicillin/Streptomycin (both Invitrogen), and 10 ng/ml recombinant GM-CSF (Peprotech, Rocky Hill, USA). Fresh medium was added to the culture on day 3, 6 and 8. On day 9, cells were harvested and plated in round-bottom 96-well plates at 2×10⁵ cells/well in 100 μl IMDM medium (Invitrogen) supplemented with 10% FBS and Penicillin/Streptomycin. Immediately after, 100 μl of IMDM medium containing the different TLR ligand mixtures were added to the cells. Medium only was added to the non-activated controls. After 24 h of incubation, 160 μl supernatant were transferred to new plates and stored at −20° C. until further analysis.

### ELISA Validation

A pilot experiment to compare the nanoimmunoassay chip to ELISA was performed by activating BM-DCs with different concentrations of single TLR ligands. BM-DCs were activated for 24 h as described above, and the secretion of IL-6 and TNFα was measured in the supernatant by ready-set-go ELISA kits (eBioscience) or by using the nanoimmunoassay chip. ELISA assay was performed according to manufacturer's instructions; plates were read on a Safire 2 microplate reader (Tecan, Männedorf, Switzerland).

1. A nanoimmunoassay chip comprising at least flow and control layers, divided into several rows, each row containing a plurality of single assay units, each assay unit contains two spotting chambers and an assay chamber in the middle, wherein neck valves separate the spotting chambers from the assay chamber during surface derivatization, said assay units being isolated from one another during incubation by isolation valves, wherein relief valves help release built-up pressure into a microfluidic channel after incubation and wherein round valves in the assay chamber define and protect the circular immunoassay regions.

2. The chip of claim 1, wherein it comprises eight rows.

3. The chip of claim 1, wherein it comprises 48 single assay units per row.

4. The chip of claim 1, wherein it comprises four round button valves.

5. The chip of claim 1, wherein each said spotting chambers has a volume of about 1.7 nL.

6. The chip of claim 1, wherein the assay chamber has a volume of about 1 nL.

7. The chip of claim 1, wherein the assay chamber comprises four circular immunoassay regions of 60 μm diameter each to detect four biomarkers of choice.

8. An analyzing system comprising at least a chip as defined in claim 1.

9. A system as defined in claim 8, further comprising at least a well plate for keeping the biological solutions, an epoxy-coated glass slide on top of which the solution is spotted and on which the chip is aligned.

10. A method of using a chip as defined in claim 1, wherein said method comprises the following steps:
a. Reagent loading.
b. Control line priming.
c. Biotin-neutravidin layer deposition.
d. Primary antibody immobilization.
e. Sample incubation.
f. Sample washing.
g. Detection step
h. Optical Readout
i. Data analysis and
l. Statistics Analysis.

11. A method comprising a chip of claim 1 which is aligned to an array of samples, or which has been directly programmed with samples using a method of sample arraying.

12. A method comprising a chip of claim 1 which is aligned to an array of detection molecules, or which has been directly programmed with an array of detection molecules including but not limited to antibodies, DNA, or aptamers.

13. A microfluidic chip of claim 1, applied to the detection/quantitation of biomarkers in biological samples, including but not limited to, blood, blood serum, BAL, cell culture medium, buffer solutions. Biomarkers include but are not limited to: proteins, peptides, DNA, RNA, organic molecules, and inorganic molecules. Biological samples may include but are not limited to: human, mouse, insect, plant, fungal, and bacterial origins.
